(19) United States
(12) Patent Application Publication
(10) Pub. No.: US 2018/0104172 A1
(43) Pub. Date: Apr. 19, 2018

(54) AIR DRY MIRROR EFFECT NAIL POLISH

(71) Applicant: MYCONE DENTAL SUPPLY CO., INC., Gibbstown, NJ (US)

(72) Inventor: Kevin SHERAN, Haddonfield, NJ (US)

(73) Assignee: MYCONE DENTAL SUPPLY CO., INC., Gibbstown, NJ (US)

(21) Appl. No.: 15/298,197

(22) Filed: Oct. 19, 2016

(51) Int. Cl. A61K 8/81; A61Q 3/02; A61K 8/36; A61K 8/35; A61K 8/73

(52) U.S. Cl. CPC A61K 8/8152; A61Q 3/02; A61K 8/36; A61K 8/35; A61K 2800/26; A61K 8/8141; A61K 2800/884; A61K 2800/43; A61K 8/731

(57) ABSTRACT

A composition for forming an air dry soft nail coating comprising an alkyl (meth)acrylate polymer and a volatile solvent after an application of the composition to a nail forms a soft nail coating. Such soft nail coating is suitable for use in preparing a mirror effect on a nail. Further, the present invention also relates to a nail coating comprising at least the soft nail coating and a powder layer. These two layers are central to a method of forming a mirror like effect on nails. A multilayer laminate nail coating comprising the bilayer, a top coat layer, and optionally a base coat layer is also taught.

### FIELD OF THE INVENTION

The present invention is directed to mirror effect coatings useful for the cosmetic adornment of fingernails. More particularly, the invention relates to the field of air dried mirror effect nail polish.

### DESCRIPTION OF RELATED TECHNOLOGY

Nails that are highly reflective, and are said to look like they are made from “metal”, “chrome”, or are “mirror like”, have become very popular over the past decade. There are various methods available to nail salon clients to obtain the mirror effect, including the use of UV/LED gel formulations, foil, and water-based products.

Aluminum containing high gloss coating for natural and artificial fingernails is disclosed in U.S. Patent Application Publication No. 2016/0007713. This publication teaches a multi-layered cosmetic laminate comprising a layer of aluminum platelets between two layers of UV cured coating.

Nail make-up composition with a mirror effect is disclosed in U.S. Patent Application Publication No. 2004/0241423. This publication teaches a composition for making up the nails, characterized in that it comprises, in a physiologically acceptable medium, particles with a metallic glint in a proportion of greater than or equal to 2% by weight, with respect to the total weight of the composition, and at least one texturizing agent or a mixture of texturizing agents in a proportion of less than or equal to 15% by weight, with respect to the total weight of the composition.

Multi-layered color-enhancing nail appliqué is taught in U.S. Pat. No. 8,905,044. A dry nail polish applique to be applied to a fingernail. The nail applique has several layers. A bottom layer hides the color of the natural nail upon which the applique is applied. A top layer of a different color is applied on top of the color-hiding layer. The color of the top layer is, as such, uninfluenced by the natural nail color and it therefore appears sharp and crisp. The top layer could be of a translucent quality such that the top layer and bottom layer visually combine to produce a unique appearance.

Mirror-like surfaces have also been generated without gels by applying and rubbing powder particles to a freshly applied quick-dry nail coat. The problem with using a currently available quick-dry nail coat in this manner is that the window during which the powder can be applied successfully is very short. If the powder is applied too early and the coat is still wet, then upon applying and rubbing the powder particles to the coat the powder particles mix with the wet coat. If the powder is applied too late and the coat is too dry, the powder does not stick to the coating.

Although many advances in the art of formulating nail polish compositions exhibiting mirror effects have been made, many more challenges remain. For example, a need continues for a method of forming an air-dry nail polish that exhibits the mirror effect.

### SUMMARY OF THE INVENTION

The present invention relates to a composition for forming an air dry soft nail coating comprising an alkyl (meth)acrylate polymer and a volatile solvent that after an application of the composition to a nail forms a soft nail coating. This soft nail coating is suitable for use in preparing a mirror effect on a nail. Further, the present invention also relates to a nail coating comprising at least the soft nail coating and a powder layer. These two layers, cumulatively a bilayer, are central to a new method of forming a mirror like effect on nails. Still further, the present invention also relates to a multilayer laminate nail coating comprising the bilayer, a top coat layer, and optionally a base coat layer.

One of the advantages of the present invention is that it provides for an easy method of using an air dry formulation to obtain a mirror-like surface. Another advantage of the present invention is that the soft coating provides a large “open time” or window in which the effects pigments can be applied to nail polish of the present invention.

The present invention is related to a fully formulated composition for forming an air dry soft nail coating comprises about 15 wt % to about 55 wt % of a poly(C₁₋₁₂alkyl (meth)acrylate) polymer; and a volatile solvent selected from the group consisting of C₁₋₄ alkyl acetate, C₁₋₃ alcohol, and C₃₋₅ ketone; wherein all wt % are with respect to the composition; wherein the ASTM D4366 Persoz pendulum hardness at dry-to-touch time is less than 20; and/or wherein the ASTM D4366 Persoz pendulum hardness 10 minutes after the dry-to-touch time is less than 100.

Under one embodiment, the poly(C₁₋₁₂alkyl (meth)acrylate) polymer is a poly(C₂₋₁₂alkyl (meth)acrylate. Under one embodiment of the present invention, poly(C₁₋₁₂alkyl (meth)acrylate) polymer comprises a poly(C₁₋₄alkyl (meth)acrylate) polymer.

In order for the air dried nail coating to be soft, the poly(C₁₋₁₂alkyl (meth)acrylate) polymer should have a relatively low glass transition temperature. The poly(C₁₋₁₂alkyl (meth)acrylate) polymers which are appropriate to form a soft have a Tg of less than about 50° C.

Alternatively, the softness of the air dried nail coating may be achieved by the use of plasticizers in an amount of about 1 wt % to about 5 wt %.

Additional optional ingredients to the composition of the present invention include additional film formers, dyes, pigments, surfactants, and their combinations.

The softness of the nail coating allows for a deeper penetration of the powder particles into the upper-most regions of the nail coating, which in turn allows for better gross adhesion of the powder particles to the soft nail coating. The Persoz pendulum hardness test, has been found to have good sensitivity in detecting differences in coating hardness.

The present invention is also directed to a nail polish midcoat bilayer comprising the soft coat layer formed from air-drying of the composition described above, and a powder layer comprising a plurality of powder particles. The powder particles as used in the present invention are comprised of a material selected from the group consisting of a metal, a metal oxide, a main group oxide, a metal/main group oxide, glitter, and a combination thereof.

The powder particles of the present invention are significantly smaller than the particles that used routinely as cosmetic glitter. The particle size needs to be sufficiently small enough to allow for working the particles onto the surface of the soft layer to produce a mirror effect, preferably, the mean particle size is less than about 60 micrometers.

The present invention is also directed to a multilayer laminate for decorating nails comprising at least the nail polish midcoat bilayer, and a top coat layer, wherein the top coat layer is on top of the nail polish midcoat bilayer. This multilayer laminate comprises at least three layers: the soft coat layer, the powder particle layer and a top coat layer.

The three-layer laminate as described above may be considered complete, and worn without any additional layers, or one or more layers may underlie the midcoat bilayer.

The present invention is also directed to a method of decorating nails comprising at least the steps of (a) applying the fully formulated nail coating composition to form a soft coat layer; and (b) applying a plurality of powder particles comprised of a material selected from the group consisting of a metal, a metal oxide, a main group oxide, a metal/main group oxide, glitter, and a combination thereof to form a powder layer. This method of decorating nail may further be followed by applying a top coat.

The present invention is also directed to a method of decorating nails comprising at least the steps of (a) applying a nail coat composition to form a base coat; (b) applying the fully formulated nail coating composition to form a soft coat layer; (c) applying a plurality of powder particles comprised of a material selected from the group consisting of a metal, a metal oxide, a main group oxide, a metal/main group oxide, glitter, and a combination thereof to form a powder layer; and (d) applying a top coat on top of the powder layer.

### BRIEF DESCRIPTION OF DRAWINGS

FIG. 1 shows photographs of the results of the application of powder particles onto samples of Examples 1 to 4, at 1.5, 2.5, 3.5, 5, 7, and 10 minutes after drawing of the formulations onto uncoated drawdown cards.

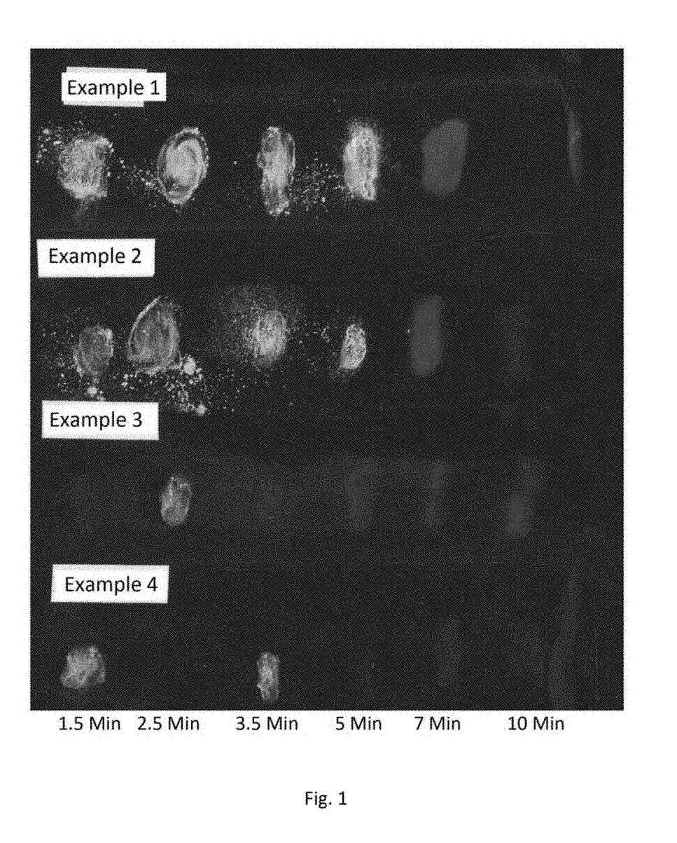

FIG. 2 shows photographs of the results of the application of powder particles onto samples of Examples 5 to 8, at 1.5, 2.5, 3.5, 5, 7, and 10 minutes after drawing of the formulations onto uncoated drawdown cards.

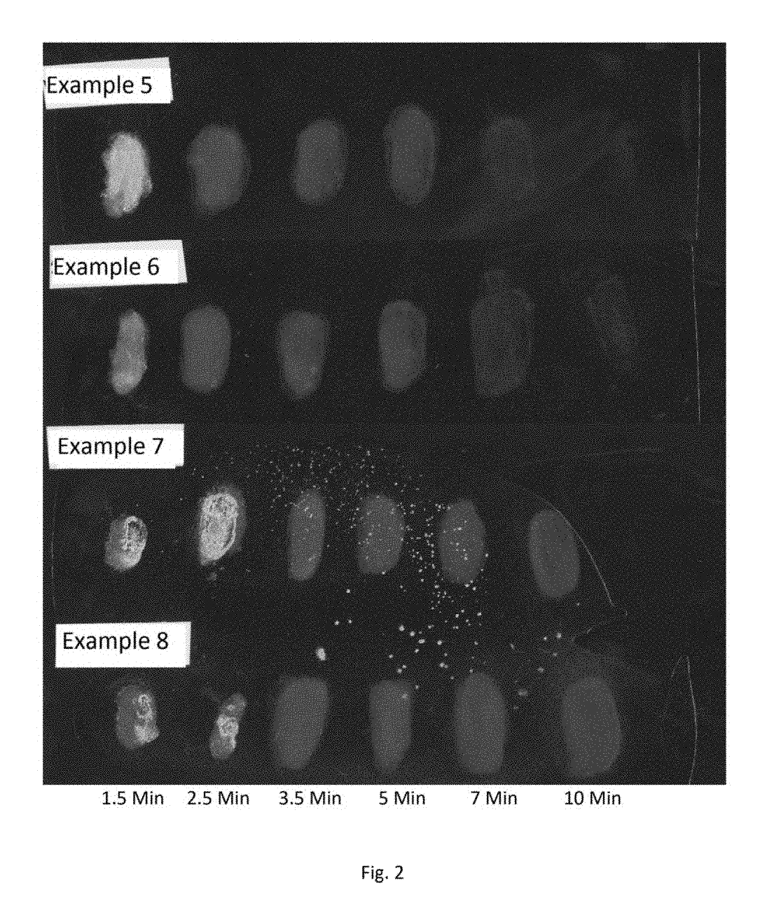

### DETAILED DESCRIPTION OF THE INVENTION

For illustrative purposes, the principles of the present invention are described by referencing various exemplary embodiments thereof Although certain embodiments of the invention are specifically described herein, one of ordinary skill in the art will readily recognize that the same principles are equally applicable to, and can be employed in other apparatuses and methods. Before explaining the disclosed embodiments of the present invention in detail, it is to be understood that the invention is not limited in its application to the details of any particular embodiment shown. The terminology used herein is for the purpose of description and not of limitation.

As used herein and in the appended claims, the singular forms “a”, “an”, and “the” include plural references unless the context clearly dictates otherwise. The singular form of any class of the ingredients refer not only to one chemical species within that class, but also to a mixture of those chemical species; for example, the term “solvent” in the singular form, may refer to a mixture of compounds each of which is also a solvent. The terms “a” (or “an”), “one or more” and “at least one” can be used interchangeably herein. It is also to be noted that the terms “comprising”, “including”, and “having” can be used interchangeably.

The term “about” when referring to a number means ±3%. For example, the phrase “about 50 wt %” refers to a number between and including 48.500 and 51.500. The term “wt %” means percent by weight.

The phrase “nail coating composition” refers to a lacquer that is suitable to be applied to fingernails or toenails to decorate or protect the nail plates, that when hardened is a nail coating.

The phrase “nail coating,” also sometimes referred to in literature as “nail polish”, “nail enamel” or “nail varnish,” refers to the hardened, fully cured substance covering a part or all of the nail, and any portions of this substance that extends or is built beyond the free edge of the nail.

The terms “nail”, refer to either a fingernail or a toenail, either a natural nail or artificial nail. The term “nail” also refers to a human nail, as well as to any toughened keratin at the end of a digit of a non-human animal.

The term “client” refers to a person whose nails are being treated.

The phrase “nail technician” or “technician” is a worker skilled or licensed in the art of providing nail extensions, artificial nails, acrylic nails, gel nails, and other manicure services for clients. Alternative names for a nail technician may include a manicurist, or a cosmetologist. Such a person may work for pay at a nail salon, or may be a manicure aficionado.

Under one embodiment of the present invention, the client and the nail technician are two different individuals. Although the description of the invention below describes the nail technician and the client as two separate individuals, it is understood that the claimed invention and methods are also suitable for use by a single person who is both a nail technician and a client. Under such embodiment of the present invention, the client and the nail technician are the same person.

The term “acrylic” in the phrase “acrylic nail” refers to hardened polymerized composition used in manicure arts, which are composed of any of several types of poly ((meth)acrylates), or copolymers of various (meth)acrylate monomers, oligomers or copolymers of various (meth)acrylate monomers with any of several non-(meth)acrylic monomers.

When referring to a composition, the definition of the term “acrylate” as referred to in the monomeric form, includes an ester, a salt, or a conjugate base of acrylic acid, with the formula CH₂═CH—COO⁻. The definition of the term “acrylate” referred to in the polymeric or oligomeric form includes the repeating unit of an ester, a salt, or a conjugate base of acrylic acid, with the formula —[CH₂—CH(COO⁻)]—.

The definition of the term “methacrylate” as referred to in the monomeric form includes an ester, a salt, or a conjugate base of methacrylic acid, with the formula CH₂═C(CH₃)—COO⁻. The definition of the term “methacrylate” as referred to in the polymeric or oligomeric form includes an ester, a salt, or a conjugate base of methacrylic acid, with the formula —[CH₂═C(CH₃)—COO⁻]—.

The term “(meth)acrylate” means acrylate, methacrylate, or a mixture thereof. When referring to a compound, “(meth)acrylate” means an ester, a salt, or a conjugate base of an acrylic acid, with the formula CH₂═C(R)—COO⁻, wherein R is H, Me, or a mixture thereof. The definition of the term “(meth)acrylate” as referred to in the polymeric or oligomeric form includes an ester, a salt, or a conjugate base of methacrylic acid, with the formula —[CH₂═C(R)—COO⁻]—, wherein R is H, Me, or a mixture thereof. By extension, a monomer, oligomer, or polymer name containing as a part of its term the string “(meth)acrylate” should be interpreted as referring to the same monomer, oligomer, or polymer, that is an acrylate, methacrylate, or a mixture thereof. For example, the term “poly(C₁₋₁₂alkyl (meth)acrylate)” means “any of poly(C₁₋₁₂alkyl acrylate), poly(C₁₋₁₂alkyl methacrylate), and a mixture of poly(C₁₋₁₂alkyl acrylate) and poly(C₁₋₁₂alkyl methacrylate)”.

The term “copolymer” means a polymer comprising more than one species of monomer. The copolymer of the present invention consists of, or comprises essentially of, one or more linear chain copolymers. Examples of a copolymer include a statistical copolymer, a random copolymer, an alternating copolymer, a periodic copolymer, and a block copolymer. Under one embodiment of the present invention, the copolymer is a statistical copolymer or a random copolymer. The term “terpolymer” is a polymer comprising three species of monomer.

The phrase “alkyl group” relates to a linear or branched saturated hydrocarbon group, which is bound to the rest of the molecule by means of a single bond. The alkyl group may contain any number of carbons that would be appropriate for use in nail polish composition. The term “alkyl group”, unless specifically referred to otherwise, may be a branched alkyl group, a linear alkyl group. The adjective form “alkyl” without a noun that it modifies following it means an “alkyl group”; likewise, the term “methyl” without a noun that it modifies following it means a “methyl group”, etc.

The abbreviation “CAP” stands for cellulose acetate propionate; “ASTM” stands for American Society for Testing and Materials; “UV” stands for ultraviolet; and “Tg” stands for the glass transition temperature.

The present invention relates to a composition for forming an air dry soft nail coating comprising an alkyl (meth)acrylate polymer and a volatile solvent that after an application of the composition to a nail forms a soft nail coating. This soft nail coating is suitable for use in preparing a mirror effect on a nail.

Further, the present invention also relates to a nail coating comprising at least the soft nail coating and a powder layer. These two layers, cumulatively a bilayer, are central to a new method of forming a mirror like effect on nails.

Still further, the present invention also relates to a multilayer laminate nail coating comprising the bilayer, a top coat layer, and optionally a base coat layer.

One of the advantages of the present invention is that it provides for an easy method of using an air dry formulation to obtain a mirror-like surface. This method avoids the need to use gel formulations and the exposure to UV lamps (in a UV-curable system method, a brush is dipped into a formulated monomer liquid, placed into the formulated polymer powder, applied to the client's nails, and after the artificial nails are formed, the artificial nails are cured under a UV lamp for several minutes). This is useful because this allows a person to apply the nail polish to herself, without the need of using UV lamps, or without visiting a nail salon.

Another advantage of the present invention that the soft coating provides a large “open time” or window in which the effects pigments can be applied compared to nail polish of the present invention. Unlike the commercially available quick-dry nail coats which have a window of about a minute or so, the window of the present invention may be several minutes, hours or longer. The reason for the long open time is that the coating is soft, and the powder adheres to it even when the coating is dry-through.

The present invention is related to a fully formulated composition for forming an air dry soft nail coating comprises about 15 wt % to about 55 wt % of a poly(C₁₋₁₂alkyl (meth)acrylate) polymer; and a volatile solvent selected from the group consisting of C₁₋₄ alkyl acetate, C₁₋₃ alcohol, and C₃₋₅ ketone; wherein all wt % are with respect to the composition; wherein the ASTM D4366 Persoz pendulum hardness at dry-to-touch time is less than 20, or the ASTM D4366 Persoz pendulum hardness 10 minutes after dry-to-touch time is less than 100.

The composition of the present invention for forming an air dry, soft nail coating comprises about 15 wt % to about 55 wt % of an alkyl (meth)acrylate polymer; and a volatile solvent. Upon application of the composition to a nail, the solvent evaporates, and the composition air dries. The air drying of the applied composition creates a soft nail coating.

The nail coating composition is a fully formulated composition, meaning that nail coating composition of the present invention is designed to be applied to the client's nails without any further addition of ingredients. Unlike the presently commercially available nail coating compositions, which are formulated to yield a hard coating upon air drying, the nail coating composition of this invention is designed to form a relatively soft coat.

The polymer is a powder and resembles the polymer powder that is typically used in the acrylic nail industry. The composition of the present invention can comprise a polyalkyl(meth)acrylate, or a mixture thereof, wherein the alkyl groups comprise one to twelve carbon atoms.

The poly(C₁₋₁₂alkyl (meth)acrylate) polymer consists of, or comprises mostly of, a polymer with repeating units —[CH₂═CR₁—COOR₂]—, wherein R₁ is either H or Me, and R₂ is an alkyl group with 1 to 12 carbons.

Under one embodiment the poly(C₁₋₁₂alkyl (meth)acrylate) polymer is a polymer prepared from one type of a monomer. Under this embodiment, the polymer consists of a single type of a polymer.

Under another embodiment the poly(C₁₋₁₂alkyl (meth)acrylate) polymer is a polymer prepared from more than one type of a monomer. Thus, a polymer of the present invention may have repeating units, wherein moieties of R₁ and R₂ of any one unit may not necessarily be the same as R₁ and R₂ of another unit. Under this embodiment the polymer may be considered a copolymer.

Under yet another embodiment, the phrase “poly(C₁₋₁₂alkyl (meth)acrylate) polymer” is a mixture of polymer, or mixture of copolymers.

Under alternative embodiments, the polymer consists of a mixture of poly(C₁₋₁₂alkyl acrylate) polymers; a mixture of poly(C₁₋₁₂alkyl methacrylate) polymers; a mixture of poly(C₁₋₁₂alkyl acrylate) polymer and poly(C₁₋₁₂alkyl methacrylate) polymer; a mixture of poly(C₁₋₁₂alkyl methacrylate) polymers and poly(C₁₋₁₂alkyl acrylate) polymer; or a mixture of poly(C₁₋₁₂alkyl methacrylate) polymers and poly(C₁₋₁₂alkyl methacrylate) polymer.

When mixed with the volatile solvent, the polymer typically forms a homogenous suspension of the polymer in the composition.

Examples of poly(C₁₋₁₂alkyl (meth)acrylate) include poly(methyl (meth)acrylate), poly(ethyl (meth)acrylate), poly(propyl (meth)acrylate), poly(n-propyl (meth)acrylate), poly(isopropyl (meth)acrylate), poly(butyl (meth)acrylate), poly(n-butyl (meth)acrylate), poly(isobutyl (meth)acrylate), poly(sec-butyl (meth)acrylate), poly(pentyl (meth)acrylate), poly(hexyl (meth)acrylate), poly(heptyl (meth)acrylate), poly(octyl (meth)acrylate), poly(nonyl (meth)acrylate), poly(decyl (meth)acrylate), poly(undecyl (meth)acrylate), and poly(dodecyl (meth)acrylate). In these examples, the alkyl groups hexyl, heptyl, octyl, nonyl, decyl, undecyl, and dodecyl may be straight chain groups or branched groups.

Examples of a branched alkyl group include: 1-methylpropyl; sec-butyl; 2-methylpropyl; iso-butyl; 1,1-dimethylethyl; tert-butyl; 1-methylbutyl; sec-pentyl; 2-methylbutyl; 3-methylbutyl; 1-ethylpropyl; 3-pentyl; 1,1-dimethylpropyl; tert-pentyl; 1,2-dimethylpropyl; 2,2-dimethylpropyl; neopentyl; 1-methylpentyl; 2-methylpentyl; 3-methylpentyl; 4-methylpentyl; iso-amyl; 1,1-dimethylbutyl; 1,2-dimethylbutyl; 1,3-dimethylbutyl; 2,2-dimethylbutyl; 2,3-dimethylbutyl, 3,3-dimethylbutyl; 1-ethylbutyl; 2-ethylbutyl; 1,1,2-trimethylpropyl; 1,2,2-trimethylpropyl; 1-ethyl-1-methylpropyl, 1-ethyl-2-methylpropyl; 1-methylhexyl; 2-methylhexyl; 3-methylhexyl; 4-methylhexyl; 5-methylhexyl; 1,1-dimethylpentyl; 1,2-dimethylpentyl; 1,3-dimethylpentyl; 1,4-dimethylpentyl; 2,2-dimethylpentyl; 2,3-dimethylpentyl; 2,4-dimethylpentyl; 3,3-dimethylpentyl; 3,4-dimethylpentyl;4,4-dimethylpentyl; 1-ethylpentyl; 2-ethylpentyl; 3-ethylpentyl; 1,1,2-trimethylbutyl; 1,1,3-trimethylbutyl; 1,2,2-trimethylbutyl; 1,2,3-trimethylbutyl; 1,3,3-trimethylbutyl; 2,2,3-trimethylbutyl; 2,3,3-trimethylbutyl; 1-(methylethyl)butyl; 1-ethyl-1-methylbutyl; 1-ethyl-3-methylbutyl; 2-(methylethyl)butyl; 2-ethyl-1-methylbutyl; 2-ethyl-2-methylbutyl; 2-ethyl-3-methylbutyl; 1-propylbutyl; 2-propylbutyl; 1,1,2,2-tetramethylpropyl; 1-ethyl-1,2-dimethylpropyl; 1-ethyl-2,2-dimethylpropyl; 1-ethyl-1,2-dimethylpropyl; 1-methylheptyl; 2-methylheptyl; 3-methylheptyl; 4-methylheptyl; 5-methylheptyl; 6-methylheptyl; 1,1-dimethylhexyl; 1,2-dimethylhexyl; 1,3-dimethylhexyl; 1,4-dimethylhexyl; 1,5-dimethylhexyl; 2,2-dimethylhexyl; 2,3-dimethylhexyl; 2,4-dimethylhexyl; 2,5-dimethylhexyl; 3,3-dimethylhexyl; 3,4-dimethylhexyl; 3,5-dimethylhexyl; 4,4-dimethylhexyl; 4,5-dimethylhexyl; 5,5-dimethylhexyl; 1-ethylhexyl; 2-ethylhexyl; 3-ethylhexyl; 4-ethylhexyl; 1,1,2-trimethylpentyl; 1,1,3-trimethylpentyl; 1,1,4-trimethylpentyl; 1,2,2-trimethylpentyl; 1,2,3-trimethylpentyl; 1,2,4-trimethylpentyl; 1,3,3-trimethylpentyl; 1,3,4-trimethylpentyl; 1,4,4-trimethylpentyl; 2,2,3-trimethylpentyl; 2,2,4-trimethylpentyl; 2,3,3-trimethylpentyl; 2,3,4-trimethylpentyl; 2,4,4-trimethylpentyl; 3,3,4-trimethylpentyl; 3,4,4-trimethylpentyl; 1-ethyl-1-methylpentyl; 1-ethyl-2-methylpentyl; 1-ethyl-3-methylpentyl; 1-ethyl-4-methylpentyl; 2-ethyl-1-methylpentyl; 2-ethyl-2-methylpentyl; 2-ethyl-3-methylpentyl; 2-ethyl-4-methylpentyl; 3-ethyl-1-methylpentyl; 3-ethyl-2-methylpentyl; 3-ethyl-3-methylpentyl; 3-ethyl-4-methylpentyl; 1-propylpentyl; 2-propylpentyl; 1-(methylethyl)pentyl; 2-(methylethyl)pentyl; 3-(methylethyl)pentyl; 1,1,2,2-tetramethylbutyl; 1,1,2,3-tetramethylbutyl; 1,1,3,3-tetramethylbutyl; 1,2,2,3-tetramethylbutyl; 1,2,3,3-tetramethylbutyl; 2,2,3,3-tetramethylbutyl; 1-ethyl-1,2-dimethylbutyl; 1-ethyl-1,3-dimethylbutyl; 1-ethyl-2,2-dimethylbutyl; 1-ethyl-2,3-dimethylbutyl; 1-ethyl-3,3-dimethylbutyl; 2-ethyl-1,1-dimethylbutyl; 2-ethyl-1,2-dimethylbutyl; 2-ethyl-1,3-dimethylbutyl; 2-ethyl-2,3-dimethylbutyl; 2-ethyl-3,3-dimethylbutyl; 1,1-diethylbutyl; 1,2-diethylbutyl; 2,2-diethylbutyl; 1-methyl-1-propylbutyl; 2-methyl-1-propylbutyl; 3-methyl-1-propylbutyl; 1-methyl-1-(methylethyl)butyl; 2-methyl-1-(methylethyl)butyl; 3-methyl-1-(methylethyl)butyl; 1-methyl-2-(methylethyl)butyl; 2-methyl-2-(methylethyl)butyl; 3-methyl-2-(methylethyl)butyl; 1-(1,1-dimethylethyl)butyl; mixtures thereof; and like. Further examples of branched alkyl groups include 8-methylnonyl, and 3,5,5-trimethylhexyl.

Although the poly(methyl(meth)acrylate) polymer may be present in a mixture of poly(C₁₋₁₂alkyl (meth)acrylate) polymers, the use of poly(methyl(meth)acrylate) polymer in a fully formulated nail coating composition formulation without any other poly(C₁₋₁₂alkyl (meth)acrylate) polymers typically yields a coating that is too hard for the powder particles to stick. To obtain soft nail coating, poly(methyl(meth)acrylate) polymer must either be mixed with polymers containing longer groups, or be augmented with plasticizers, or a combination thereof.

Under one embodiment, the poly(C₁₋₁₂alkyl (meth)acrylate) polymer is a poly(C₂₋₁₂alkyl (meth)acrylate.

Under one embodiment of the present invention, poly(C₁₋₁₂alkyl (meth)acrylate) polymer comprises a poly(C₁₋₄alkyl (meth)acrylate) polymer. Poly(C₁₋₄alkyl (meth)acrylate) is a polymer comprising —[CH₂═CR₁—COOR₂]— repeating units, wherein R₁ is either H or Me, and R₂ is an alkyl group with one to four carbons. Examples of poly(C₁₋₄alkyl (meth)acrylate) include poly(methyl (meth)acrylate), poly(methyl acrylate), poly(methyl methacrylate), poly(ethyl (meth)acrylate), poly(ethyl acrylate), poly(ethyl methacrylate), poly(propyl (meth)acrylate), poly(propyl acrylate), poly(propyl methacrylate), poly(butyl (meth)acrylate), poly(butyl acrylate), and poly(butyl methacrylate)

Further, the definition of poly(C₁₋₄alkyl (meth)acrylate) also includes the copolymers of poly(C₁₋₄alkyl (meth)acrylate). Under this embodiment, the poly(C₁₋₄alkyl (meth)acrylate) polymer is a copolymer prepared from one type of a monomer.

Under another embodiment the poly(C₁₋₄alkyl (meth)acrylate) polymer is a polymer prepared from more than one type of a monomer. Thus, a polymer of the present invention may have repeating units —[CH₂═CR₁—COOR₂]—, wherein moieties of R₁ and R₂ of any one unit may not necessarily be the same as R₁ and R₂ of another unit.

Under yet another embodiment, the phrase “poly(C₁₋₄alkyl (meth)acrylate) polymer” is a mixture of polymer, or mixture of copolymers.

The above poly(C₁₋₁₂alkyl (meth)acrylate) polymer or the poly(C₁₋₄alkyl (meth)acrylate) polymer may be obtained from any number of commercial sources, including Dow (as PARALOID™), Evonik (as Degalan®), and like.

In order for the air dried nail coating to be soft, the poly(C₁₋₁₂alkyl (meth)acrylate) polymer should have a relatively low glass transition temperature. The glass transition temperature of the poly(C₁₋₁₂alkyl (meth)acrylate) polymer depends on various factors, such as the identities of R₁, R₂, molecular weight, molecular weight distribution, tacticity, thermal history, purity, and the method of measurement.

The use of polymers exhibiting low glass transition temperatures tend to result in softer coatings. The use of polymers exhibiting high glass transition temperatures tend to result in harder coatings

The poly(C₁₋₁₂alkyl (meth)acrylate) polymers which are appropriate to form a soft have a Tg of less than about 50° C.

The fully formulated nail coating composition of the present invention also comprises a volatile solvent selected from the group consisting of C₁₋₄ alkyl acetate, C₁₋₃ alcohol, C₃₋₅ ketone, and a mixture thereof.

The solvent C₁₋₄ alkyl acetate is a composition that consists of, consists essentially of, or comprises mostly of a compound of formula CH₃—C(O)—OR′, wherein R′ is a —CH₃, methyl, —CH₂CH₃, ethyl, —CH₂CH₂CH₃, n-propyl, —CH(CH₃)₂, i-propyl, —CH₂CH₂CH₂CH₃, n-butyl, —CH(CH₃)CH₂CH₃, sec-butyl, —CH₂CH(CH₃)₂, isobutyl, —C(CH₃)₃, tert-butyl, and mixtures thereof.

The solvent C₁₋₃ alcohol is a composition that consists of, consists essentially of, or comprises mostly of a compound of formula R′OH, wherein R′ is a —CH₃, methyl, —CH₂CH₃, ethyl, —CH₂CH₂CH₃, n-propyl, —CH(CH₃)₂, i-propyl, and mixtures thereof.

The solvent C₃₋₅ ketone is a composition that consists of, consists essentially of, or comprises mostly of a compound of formula R′—C(O)—R″, wherein R′ is a —CH₃, or —CH₂CH₃, R″ is —CH₃, —CH₂CH₃, —CH₂CH₂CH₃, or —CH(CH₃)₂ when R′ is —CH₃; and R″ is —CH₃, or —CH₂CH₃, when R′ is —CH₂CH₃; and mixtures thereof.

Examples of volatile solvents include methanol, ethanol, isopropanol, n-butanol, s-butanol, i-butanol, methanol, ethanol, propanol, isopropanol, acetone, methyl ethyl ketone, butanone, methyl propyl ketone, 2-pentanone, diethyl ketone, 3-pentanone, methyl isopropyl ketone, 3-methylbutan-2-one, and mixtures thereof.

Aside from selecting polymers with long alkyl groups, and polymers with low Tg, softness of the air dried nail coating may alternatively or additionally be obtained by the addition of at least one plasticizer. The plasticizer are used to decrease the hardness of the resulting soft coating.

Examples of plasticizers includes acetyltributylcitrate, camphor, dibutyl oxalate, dibutyl phthalate, dibutyl sebacate, diethyl oxalate, diethyl phthalate, diethyl sebacate, diethyl succinate, dimethyl phthalate, dioctyl adipate, dioctyl phthalate, n-decane, neopentyl glycol, o-toluenesulfonamide, p-toluenesulfonamide, epoxidized soybean oil, triacetin, tributyl citrate, triethyl citrate, tri(2-ethylhexyl) trimellitate, 2,2,4-trimethyl-1,3-pentanediol dibenzoate, 2,2,4-trimethyl-1,3-pentanediol diisobutyrate, triphenyl phosphate, tritolyl phosphate, and mixtures thereof.

The plasticizer may be employed in the fully formulated nail coating composition for forming an air dry soft nail coating in an amount of about 1 wt % to about 5 wt %. Alternatively, the plasticizer is employed in the composition in an amount of about 1 wt % to about 3 wt %.

In addition to the poly(C₁₋₁₂alkyl (meth)acrylate) polymer and the volatile solvent, the fully formulated nail coating composition for forming an air dry soft nail coating may further comprise other ingredients. Such ingredients may include one or more of the following ingredients: an additional film former, a dye, a pigment, a surfactant, and a combination thereof.

Any additional ingredient may be included in the fully formulated nail coating composition of the present invention, as long as the fully formulated nail coating composition upon air drying forms a soft nail coating.

Examples of additional film formers include alkyl alkacrylates, cellulose acetate, cellulose acetate butyrate, cellulose acetate propionate, ethyl cellulose, vinyl polymers, nitrocellulose, and mixtures thereof.

An additional ingredients to the fully formulated nail coating composition of the present invention under one embodiment include a small amount of a colorant or a pigment.

One purpose of using pigment in the composition of the present invention is to provide a tint or a color to the formed soft nail coating. The exhibition of such a tint or a color may provide a contrasting background onto which the powdered layer is applied. Under this embodiment, the contrast between the soft nail coating and the powder allows the technician to apply an even coat of powder. For example, an area of a nail has the hue of the soft nail coating may be deemed to have too little powder coating.

Another purpose of using a pigment is to provide a whitish appearance to the photopolymerizable composition, so that it appears as an attractive, clean product to the nail technician.

Examples of pigments may be incorporated into the composition of the present invention include: annatto, caramel, carmine, B-carotene, potassium sodium copper chlorophyllin (chlorophyllin copper complex), dihydroxyacetone, bismuth oxychloride, guaiazulene, iron oxides, ferric ammonium ferrocyanide, ferric ferrocyanide, chromium hydroxide green, chromium oxide greens, guanine, pyrophyllite, mica, silver, titanium dioxide, aluminum powder, bronze powder, copper powder, ultramarines, manganese violet, zinc oxide, luminescent zinc sulfide, FD&C Blue No. 1, D&C Blue No. 4, Iron Blue, D&C Brown No. 1, FD&C Green No. 3, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, FD&C Red No. 40, D&C Violet No. 2, Ext. D&C Violet No. 2, FD&C Yellow No. 5, FD&C Yellow No. 6, D&C Yellow No. 7, Ext. D&C Yellow No. 7, D&C Yellow No. 8, D&C Yellow No. 10, D&C Yellow No. 11, and mixture of any of the foregoing.

Upon the application of the fully formulated nail coating composition of the present invention onto a nail or another nail coat, the composition air dries to form a soft nail coating.

The nail coating is deemed to be “dry” when that nail coating is dry-to-touch, as defined by ASTM method D 1640-03. Under such method, the film is touched lightly at varying intervals of time. The film is considered dry to touch when no pronounced marks are left by the finger touching the film in the same area on each observation.

The nail coating of the present invention dries within about five minutes. Under another embodiment, the nail coating dries within about 3 minutes. Under yet another embodiment, the nail coating dries within about 1 minute.

Under another embodiment, the nail coating is deemed to be “dry” when that nail coating is through dry. A nail coating is through dry, when a thumb or another digit, is pressed firmly (at about 100 kPa pressure) onto a surface of the nail coating, the thumb is turned about 90° in 1 second, and there is no visible change to the coating.

It is hypothesized that the softness of the nail coating allows a deeper penetration of the powder particles into the upper-most regions of the nail coating, thus increasing the area of the interaction of the powder particles with the nail coating, and thus allowing for better gross adhesion of the powder particles to the soft nail coating.

Less than 5 minutes after the application of the fully formulated nail coating composition of the present invention, the resulting nail coating is dry.

Under one embodiment of the present invention, the Persoz pendulum hardness of the formed nail coat at dry-to-touch dryness is less than 20 as measured by ASTM D4366. The phrase “at the dry-to-touch time” means that the determination of the Persoz pendulum hardness begins immediately after the dry-to-touch time.

Under another embodiment of the present invention, the Persoz pendulum hardness of the formed nail coat at 10 minutes after the dry-to-touch dryness is less than 100 as measured by ASTM D4366. The phrase “at 10 minutes after the dry-to-touch time” means that the determination of the Persoz pendulum hardness begins to be determined 10 minutes after the observance of the dry-to-touch time.

Under yet another embodiment of the present invention, the Persoz pendulum hardness of the formed nail coat at dry-to-touch dryness is less than 20 as measured by ASTM D4366; and the Persoz pendulum hardness of the formed nail coat at 10 minutes after dry-to-touch dryness is less than 100 as measured by ASTM D4366.

The examples 2, and 5 to 8 discussed further below, have both Persoz pendulum hardness at less than 20 at dry-to-touch dryness, and have Persoz pendulum hardness of the formed nail coat at 10 minutes after dry-to-touch dryness of less than 100.

The Persoz pendulum hardness test, as tested by using ASTM standard D4366-14, has been found to have good sensitivity in detecting differences in coating hardness, where hardness is defined as resistance to deformation.

It is also an aspect of the present invention that upon air drying of the fully formulated nail coating composition, the formed nail coat remains soft for at least 30 minutes. Under another embodiment, the nail coat remains soft for at least 4 hours.

About 30 minutes after the application of the fully formulated nail coating composition of the present invention, the resulting nail coating has a Persoz pendulum hardness of less than 100. Under one embodiment, the resulting coating has a Persoz pendulum hardness of less than 50.

The present invention is also directed to a nail polish midcoat bilayer comprising the soft coat layer formed from air-drying of the composition described above, and a powder layer comprising a plurality of powder particles comprised of a material selected from the group consisting of a metal, a metal oxide, a main group oxide, a metal/main group oxide, glitter, and a combination thereof; wherein the powder particles of the powder layer adhere to the top surface of the soft coat layer with a force of less than twice the gravitational pull.

The term “midcoat” as used in the coating industry, including automotive and motorcycle coatings, refers to the coat which includes particles (such as metal flakes and dry pearls) which give the multicoat laminate a special color effect. Under one embodiment, the midcoat of the present invention is a part of a multilayer laminate, which further includes a top coat layer, and optionally a base layer, and optionally further layers. Under another embodiment, the midcoat can be applied to nails and worn without any further coatings.

Unlike for automobile coatings, where the powder particles are mixed into the midcoat, in the present invention the powder is added after the application and drying of the soft layer. The sequential application of the powder after the soft coat layer results in a bilayer.

The midcoat of the present invention is a bilayer, comprising a soft coat bottom layer (the layer closer to the nail), and a powder particle top layer (the layer further away from the nail than the bottom layer). The soft coat layer is formed from air drying the above-described fully formulated nail coating composition for forming an air dry soft nail. The powder particle top layer is comprised of a plurality of powdered particles.

The powder particles as used in the present invention are comprised of a material selected from the group consisting of a metal, a metal oxide, a main group oxide, a metal/main group oxide, glitter, and a combination thereof.

The material from which the powder particles are made is nonreactive with the polymers that make up the soft bottom layer, nor with polymers which may be used as a top layer. Non-reactive means that there is no chemical reaction at all, or that there is no appreciable reaction.

Further, the material should be non-toxic at the levels to the client and/or to the nail technician are exposed to powder when the powder is applied or when worn.

Examples of metals include transition metals and main group metals. Examples of suitable transition metals include titanium, chromium, iron, cobalt, nickel, palladium, platinum, copper, silver, gold, and zinc. Examples of main group metals include aluminum, tin, and bismuth. Suitable metals include coinage metals or precious metals. Further, particularly suited materials include silver and aluminum.

Additionally, metals also include alloys of any transition metal or a main group metal with other transition metals or main group metals.

Metal oxides include both transition metal oxides and main group metal oxides, such as tin oxide, iron oxide and aluminum oxides.

Metal oxides include main group metal oxides, main group non-metal oxides, and a combination thereof. Examples include silicon oxide, glass, aluminum oxide, tin oxide, calcium aluminum borosilicate, and bismuth oxychloride.

The term “glitter” means any material which may be used in the cosmetic industry, the particles of which reflect light in various angles, causing the surface comprising such material to sparkle or shimmer. Glitters are small flat reflective particles are typically prepared from thin plastic sheets coated with a reflective material. The composition of glitter include a polymer and any of a metal, a metal oxide, a main group oxide, a metal/main group oxide, or a combination thereof.

Additional suitable materials from which powder particles are comprised of include a combination material of any of the foregoing. Examples include phyllosilicate, synthetic fluorphlogopite, mica, and other minerals, such as biotite, lepidolite, phlogopite, muscovite, clintonite, and zinnwaldite.

Examples of phyllosilicate include calcium sodium borosilicate, calcium aluminum borosilicate, calcium titanium borosilicate, silver borosilicate, and zinc borosilicate.

The powder particles of the present invention are significantly smaller than the particles that used routinely as cosmetic glitter. The particle size needs to be sufficiently small enough to allow for working the particles onto the surface of the soft layer to produce a mirror effect. Preferably, the mean particle size is less than about 60 micrometers. If the particles are glitter, preferably the mean particle size is less than about 60 micrometers.

The powder particles may have any shape, as deemed by the physical characteristics (such as a diffraction pattern, natural cleavage of minerals) of the material. It was observed that spherical borosilicate glass appears to provide better results in sticking to the soft coat than powder particles of other shapes.

When the powder particles are applied to the surface of the soft coating, the particles at first simply lie on top of the surface. Working the particles down into the soft layer with a tool (such as an eye makeup applicator, a cotton swab, or a finger) forces pressure onto the particles, which is hypothesized to indent the upper surface of the soft coat with the powder particles.

After the working of the particles, a significant portion of the particles sticks to the soft layer. Some of the excess particles may be shaken off. Shaking off the particles, achieved by a rapid movement of the hand of the client, or by shaking off the hand of the client. This shaking of the powder particles exposes the powder particles to acceleration of about twice the gravitational pull, or about 2 g-forces, or about 20 m·s⁻². The particles that can be shaken off stick with a force of less than about 2 g, whereas the powder particles of the powder layer which cannot be easily shaken off adhere to the top surface of the soft coat layer with a force of greater than twice the gravitational pull.

Alternatively to shaking of the excess powder particles, the excess powder particles may be bushed off with a cosmetic brush such as makeup brushes. The excess powder particles are brushed off with much less force then is the force used to work the powder particles onto the surface of the soft coating.

The present invention is also directed to a multilayer laminate for decorating nails comprising at least the nail polish midcoat bilayer, and a top coat layer, wherein the top coat layer is on top of the nail polish midcoat bilayer.

This multilayer laminate comprises at least three layers: the soft coat layer, the powder particle layer and a top coat layer.

The phrases “top coat” and “top coat layer” refer to the coating that lies immediately atop of the powder particle layer. In addition to the top coat layer, there may be more layers applied on top of the top coat.

The top coat layer may also be an air dry layer. Alternatively, the top coat layer may be cured UV radiation.

Under one embodiment, the top coat and any other coats over the powder particle layer are free of pigment so that the mirror appearance is clearly visible. Under another embodiment, the top coat or any other subsequent coats contain a small amount of pigment to yield a tinted top coat.

The three-layer laminate as described above may be considered complete, and worn without any additional layers.

Alternatively, one or more layers may underlie the midcoat bilayer. Under this embodiment, the multilayer laminate contains at least 4 layers: a base layer, a soft layer, a powder particle layer, and a top coat layer.

The term “base layer” as used herein refers to the layer immediately underlying the midcoat bilayer. The base layer may be a color layer, or a sealant layer. Underneath the base layer, there may be additional layers.

The bottom coat layer may also be an air dry layer. Alternatively, the bottom coat layer may be cured by UV radiation.

The present invention is also directed to a method of decorating nails comprising at least the steps of (a) applying the fully formulated nail coating composition to form a soft coat layer; and (b) applying a plurality of powder particles comprised of a material selected from the group consisting of a metal, a metal oxide, a main group oxide, a metal/main group oxide, glitter, and a combination thereof to form a powder layer.

This method involves applying the previously defined fully formulated nail coating composition directly to the nail or any other substrate, waiting for the fully formulated nail coat to dry, and applying the powder layer. The application of the fully formulated nail coating composition is may be done by applying the nail coating composition with a nail polish brush, or by any other manner to achieve a uniform coating.

The powder particles are applied in any manner which transfer a plurality of powder particle onto a nail. This may include dipping the nail into a vessel containing the powder, or transferring the powder from the vessel onto the finger with an applicator, such as a makeup applicator.

Optionally, after the powder is transferred onto the nail, the applicator (or any other tool, such as a small brush, a cotton swab or a finger) is used to spread the powder around, and to work the particles into the soft layer. Working the particles means pushing on the particles and swirl them in repetitive motion, such as circular motion or linear motion.

After a sufficient amount of work on the particles, the powder layer achieves a uniform mirror-like finish. The phrase “mirror-like” or “mirror effect” means that the nail reflects most of the light, like a mirror does. Another term for “mirror-like” or “mirror effect” is “chrome” or “metallic”.

This method of decorating nail may further be followed by applying a top coat. This application may be done by applying the top coat composition with a nail polish brush, or by any other manner to achieve a uniform coating.

The present invention is also directed to a method of decorating nails comprising at least the steps of (a) applying a nail coat composition to form a base coat; (b) applying the fully formulated nail coating composition to form a soft coat layer; (c) applying a plurality of powder particles comprised of a material selected from the group consisting of a metal, a metal oxide, a main group oxide, a metal/main group oxide, glitter, and a combination thereof to form a powder layer; and (d) applying a top coat on top of the powder layer.

These four steps are done in a sequential manner. Additional steps may take place between any two steps. For example, after the application of the powder particles in step (c), a step of working the particles onto the surface of the soft coat layer may optionally precede step (d).

The amounts of base coat composition, fully formulated nail coating composition for soft coat, and top coat are selected to produce a dried coating thickness of about 0.001 inches to about 0.005 inches (about 25 μm to 125 μm), preferably about 0.002 inches to 0.003 inches (50 μm to 75 μm).

### EXPERIMENTAL

### Materials

PARALOID™ B-66 100% is a solid grade thermoplastic acrylic resin, with a methyl methacrylate/butyl methacrylate base composition, with a mean molecular weight of 70,000 g/mol, and a glass transition temperature of 50° C. When suspended in appropriate solvents, it forms clear solutions that air-dry to form colorless films with a Knoop hardness of about 12 to 13.

PARALOID B-99N 100% is a solid grade thermoplastic acrylic resin, with a methyl methacrylate/butyl methacrylate base composition, with a mean molecular weight of 15,000 g/mol, and a glass transition temperature of 82° C. When suspended in appropriate solvents, it forms clear solutions that air-dry to form colorless films with a Knoop hardness of about 14 to 15.

PARALOID A-101 100% is a solid grade thermoplastic acrylic resin, with a methyl methacrylate base composition, with a mean molecular weight of 100,000 g/mol, and a glass transition temperature of 105° C. When suspended in appropriate solvents, it forms clear solutions that air-dry to form films with a Knoop hardness of about 21 to 22.

PARALOID A-101 40% is a 5,000 cP solution comprising 60% methyl ethyl ketone and 40% methyl methacrylate with a mean molecular weight of 100,000 g/mol and a glass transition temperature of 105° C. When suspended in appropriate solvents, it forms clear solutions that air-dry to form films with a Knoop hardness of about 21 to 22.

PARALOID B-72 100% is a solid grade thermoplastic acrylic resin, with an ethyl methacrylate base composition, with a mean molecular weight of 105,000 g/mol, and a glass transition temperature of 40° C. When suspended in appropriate solvents, it forms clear solutions that air-dries to form colorless films with a Knoop hardness of about 10 to 11.

CN9064 (Pro 12275) is a trifunctional aliphatic urethane acrylate oligomer, available from Sartomer. This oligomer is based on H12MDI and a polypropylene glycol core.

### Mid Coat Nail Polish Formulation

The formulations of eight Examples are disclosed in Table 1. Examples 1, 3, and 4 are Comparative Examples, and Examples 2, and 5 to 8 are Working Examples.

TABLE 1
Mid-coat Formulations

| | | Examples | | | | | | | |
|---|---|---|---|---|---|---|---|---|---|
| Ingredients | T_{g}, ° C. | 1 | 2 | 3 | 4 | 5 | 6 | 7 | 8 |
| Acetate solvent | | 77.2 | 69.0 | 50.2 | 50.2 | 79.9 | 50.0 | 70.0 | 60.0 |
| Methyl ethyl ketone | | | | | 29.9 | | 30.0 | | |
| CAP | | 12.1 | 10.9 | | | | | | |
| Paraloid B-72 100% | 40 | | | | | 20.1 | 20.1 | 30.0 | 40.0 |
| Paraloid B-66 100% | 50 | 8.3 | 7.4 | | | | | | |
| CN9064 (Pro 12275) | 72 | | 10.6 | | | | | | |
| Paraloid B-99N 100% | 80 | 2.3 | 2.0 | | | | | | |
| Paraloid A-101 100% | 105 | | | | 19.9 | | | | |
| Paraloid A-101 40% | 105 | | | 49.8 | | | | | |
| Dyes, surfactants | | 0.1 | 0.1 | | | | | | |

For each of the eight examples of the above prepared formulations six samples ovals approximating a nail were applied onto uncoated drawdown cards (byko-chart N9A, available from BYK-Gardner GmbH) using standard procedures. Powder particles were added after 1.5, 2.5, 3.5, 5, 7 and 10 minutes using internally consistent procedures. The powder particles were added regardless of the drying times of the formulations.

Comparative Example 1 shows splotchy bilayers and loose powder particles at 1.5, 2.5, 3.5, and 5 minutes, indicating that the powder particles mixed with the wet samples of the formulation. The sample at 7 minutes showed a good coverage of the powder particles onto the formulation of Example 1. However, the 10 minute sample showed no adhesion of the powder particles onto the formulation.

The samples of Working Example 2 exhibits similar behavior as that for Comparative Example 1, except that the 10 minute sample showed adhesion of the powder particles onto the formulation.

Comparative Examples 3 and 4 show splotchy bilayer and loose powder particles, and very faint or non-existent adhesion of the powder particles onto the formulation for 5, 7, and 10 minute samples. There very faint or non-existent showings of adhesion are indicative of the hardness being too high to allow adherence to the formulations.

Working Examples 5 and 6 show splotchy bilayer and loose powder at 1.5 minutes. The samples for 2.5, 3.5, 5, 7, and 10 minutes show a smooth, uniform bilayer, indicating significant adhesion of the powder particles to the samples. Further, the later samples are progressively fainter, indicating hardening of the mid-coat formulation.

Working Examples 7 and 8 show splotchy bilayer and loose powder at 1.5 and 2.5 minutes, indicating that the samples were still wet. The samples for 3.5, 5, 7, and 10 minutes show a significant adhesion of the powder particles to the samples. Unlike for Working Examples 5 and 6, each of the 3.4, 5, 7, and 10 minute samples for Examples 7 and 8 appear to have approximately the same albedo, indicating that the formulations remained soft for at least 10 minutes after the application.

Additionally, the drying time for each of the eight formulations was measured on new samples. After about 3 minutes, Examples 3 to 8 were found to have air dried to be dry to touch. Examples 2, and 5 to 8 were judged to be soft enough to accept aluminum powder readily.

The dry-to-touch drying time was determined by the ASTM method D 1640-2003.

The hardness of the composition was determined via Persoz pendulum test using the ASTM method D4366-2014. The results of this test are shown in Table 2.

TABLE 2
Persoz pendulum hardness test

| | Examples | | | | | | | |
|---|---|---|---|---|---|---|---|---|
| | 1 | 2 | 3 | 4 | 5 | 6 | 7 | 8 |
| 5 min | | 10 | 80 | 80 | 11 | 14 | 14 | 8 |
| 15 min | | 29 | 222 | | 33 | 34 | 17 | 18 |
| 30 min | | 47 | 210 | | 38 | 40 | 25 | 20 |
| 45 min | | 51 | 227 | | 35 | 39 | 25 | 20 |
| 1 hour | 295 | 63 | 229 | | 52 | 49 | 24 | 22 |
| 2 hour | | 84 | 228 | | 87 | 61 | 28 | 28 |
| 3 hour | | 80 | 231 | | 97 | 82 | 51 | 29 |
| 4 hour | | 91 | 230 | | 102 | 97 | 45 | 32 |

Working Examples 5 and 6 showed that the identity of the solvent has little influence on the hardness of the formed soft coat. Further, Working Examples 5, 7, and 8, show that higher amounts of the polymer yield a soft coat which maintains the softness for a longer amount of time.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1. A composition comprising
(i) about 15 wt % to about 55 wt % of a poly(C₁₋₁₂alkyl (meth)acrylate) polymer; and
(ii) a volatile solvent selected from the group consisting of C₁₋₄ alkyl acetate, C₁₋₃ alcohol, C₃₋₅ ketone, and mixtures thereof;
wherein all wt % are with respect to the composition;
wherein the ASTM D4366 Persoz pendulum hardness at the dry-to-touch time is less than 20; or wherein the ASTM D4366 Persoz pendulum hardness 10 minutes after the dry-to-touch time is less than 100;
wherein the composition is fully formulated nail coating composition for forming an air dry soft nail coating.

2. The composition of claim 1, wherein the Persoz pendulum hardness at the dry-to-touch time is less than 20.

3. The composition of claim 1, wherein the Persoz pendulum hardness 10 minutes after at the dry-to-touch time is less than 100.

4. The composition of claim 1, wherein the Persoz pendulum hardness at the dry-to-touch time is less than 20, and wherein the Persoz pendulum hardness 10 minutes after at the dry-to-touch time is less than 100.

5. The composition of claim 1, wherein the glass transition temperature of the poly(C₁₋₁₂alkyl (meth)acrylate) polymer is less than 50° C.

6. The composition of claim 1, wherein the poly(C₁₋₁₂alkyl (meth)acrylate) polymer is poly(C₂₋₁₂alkyl (meth)acrylate).

7. The composition of claim 1, wherein the poly(C₁₋₁₂alkyl (meth)acrylate) comprises a poly(C₁₋₄alkyl (meth)acrylate) selected from the group consisting of poly(ethyl (meth)acrylate), poly(propyl (meth)acrylate), poly(butyl (meth)acrylate), copolymers thereof, and mixtures thereof.

8. The composition of claim 1, wherein the composition further comprises at least one plasticizer selected from the group consisting of acetyltributylcitrate, camphor, dibutyl oxalate, dibutyl phthalate, dibutyl sebacate, diethyl oxalate, diethyl phthalate, diethyl sebacate, diethyl succinate, dimethyl phthalate, dioctyl adipate, dioctyl phthalate, n-decane, neopentyl glycol, o-toluenesulfonamide, p-toluenesulfonamide, epoxidized soybean oil, triacetin, tributyl citrate, triethyl citrate, tri(2-ethylhexyl) trimellitate, 2,2,4-trimethyl-1,3-pentanediol dibenzoate, 2,2,4-trimethyl-1,3-pentanediol diisobutyrate, triphenyl phosphate, tritolyl phosphate, and mixtures thereof.

9. A nail polish midcoat bilayer comprising
(a) a soft coat layer formed from air-drying of the composition of claim 1, comprising a top surface and a bottom surface, and
(b) a powder layer comprising a plurality of powder particles comprised of a material selected from the group consisting of a metal, a metal oxide, a main group oxide, a metal/main group oxide, glitter, and a combination thereof;
wherein the powder particles of the powder layer adhere to the top surface of the soft coat layer with a force of greater than twice the gravitational pull.

10. The midcoat bilayer of claim 9, wherein the material is selected from the group consisting of aluminum, phyllosilicate, borosilicate glass, tin oxide, silicon oxide, and synthetic fluorphlogopite.

11. The midcoat bilayer of claim 9, wherein the material is phyllosilicate selected from the group consisting of calcium sodium borosilicate, calcium aluminum borosilicate, calcium titanium borosilicate, silver borosilicate, and zinc borosilicate.

12. The midcoat bilayer of claim 9, wherein the material is metal selected from the group consisting of aluminum and silver.

13. The midcoat bilayer of claim 9, wherein the mean particle size of the powder particles is less than about 60 micrometers.

14. The midcoat bilayer of claim 9, wherein the material is glitter, and wherein the mean particle size of the powder particles is less than about 60 micrometers.

15. A multilayer laminate for decorating nails comprising at least the nail polish midcoat bilayer of claim 9, and a top coat layer, wherein the top coat layer is on top of the nail polish midcoat bilayer.

16. The multilayer laminate of claim 15, wherein the multilayer laminate further comprises a base coat layer, wherein the base coat layer underlies the midcoat bilayer.

17. A method of decorating nails comprising at least the steps of
(a) applying a fully formulated nail coating composition for forming an air dry soft nail coating comprising (i) about 15 wt % to about 55 wt % of a poly(C₁₋₁₂alkyl (meth)acrylate) polymer; and (ii) a volatile solvent selected from the group consisting of C₁₋₄ alkyl acetate, C₁₋₃ alcohol, C₃₋₅ ketone, and mixtures thereof; wherein all wt % are with respect to the composition; and wherein the ASTM D4366 Persoz pendulum hardness at the dry-to-touch time is less than 20, or wherein the ASTM D4366 Persoz pendulum hardness 10 minutes after the dry-to-touch time is less than 100; to form a soft coat layer; and
(b) applying a plurality of powder particles comprised of a material selected from the group consisting of a metal, a metal oxide, a main group oxide, a metal/main group oxide, and a combination thereof to form a powder layer.

18. The method of claim 17, wherein the step (b) is followed by a step of applying a top coat composition on top of the powder layer.

19. A method of decorating nails comprising at least the steps of
(a) applying a nail varnish composition to form a base coat;
(b) applying a fully formulated nail coating composition for forming an air dry soft nail coating comprising (i) about 15 wt % to about 55 wt % of a poly(C₁₋₁₂alkyl (meth)acrylate) polymer; and (ii) a volatile solvent selected from the group consisting of C₁₋₄ alkyl acetate, C₁₋₃ alcohol, C₃₋₅ ketone, and mixtures thereof; wherein all wt % are with respect to the composition; and wherein the ASTM D4366 Persoz pendulum hardness at the dry-to-touch time is less than 20; or wherein the ASTM D4366 Persoz pendulum hardness 10 minutes after the dry-to-touch time is less than 100; to form a soft coat layer;
(c) applying a plurality of powder particles comprised of a material selected from the group consisting of a metal, a metal oxide, a main group oxide, a metal/main group oxide, glitter, and a combination thereof to form a powder layer; and
(d) applying a top coat on top of the powder layer.
